(19) United States
(12) Patent Application Publication
(10) Pub. No.: US 2008/0243166 A1
(43) Pub. Date: Oct. 2, 2008

(54) Pouch-Equipped Intragastric Balloon

(75) Inventors: Pascal Paganon, Serezin du Rhone (FR); Jean-Paul Gilbert Ricol, Saint Germaine sur I'Arbresle (FR)

Correspondence Address: POWELL GOLDSTEIN LLP, ONE ATLANTIC CENTER FOURTEENTH FLOOR, 1201 WEST PEACHTREE STREET NW, ATLANTA, GA, 30309-3488, US

(73) Assignee: COMPAGNIE EUROPEENNE D'ETUDE ET DE RECHERCHE DE DISPOSITIFS POUR I'IMPLANTATION PAR LAPAROSCOPIE

(21) Appl. No.: 10/598,092

(22) PCT Filed: Feb. 18, 2005
(86) PCT No.: PCT/FR2005/000388

§ 371 Date: Apr. 9, 2008

(30) Foreign Application Priority Data
Feb. 20, 2004 (FR) 0401730

(51) Int. Cl. A61M 29/02; B23P 11/00

(52) U.S. Cl. 606/192; 29/428

(57) ABSTRACT

An expandable intragastric balloon comprising a first flexible pouch (2) comprising a port; a second pouch (20) which contains the first pouch (2) and which is equipped with a hole (21); and, a sealing member (4) which is hermetically fixed to the second pouch (20) and which is intended to seal the port and the hole (21). The first and second pouches (2, 20) are produced from different, non-compatible materials and are assembled with the aid of a fixing element (7). The fixing element is designed to ensure that the sealing member (4) is hermetically fixed to the first pouch (2) inside a passage (6) that is defined by a neck (5) which extends from the port, such that sufficient pressure is exerted on the neck (5) in order for same to be pinched between the sealing member (4) and the fixing element (7).

### PRIORITY CLAIM

This patent application is a U.S. National Phase of International Application No. PCT/FR2005/000388, having an International Filing Date of Feb. 18, 2005, which claims priority to French Patent Application No. FR 04 01730, having a Filing Date of Feb. 20, 2004, the disclosures of which are incorporated herein by reference in their entirety.

### FIELD OF THE INVENTION

This invention relates to devices that can be implanted in the human body and that are designed to be used for the treatment of obesity, and particularly morbid obesity, and most particularly to implants capable of reducing the volume of the stomach for the particular purpose of producing a sensation of fullness in the patient.

This invention relates to an expandable intragastric balloon designed to be implanted inside the stomach of a patient for the treatment of obesity, and comprising:
a first pouch that is sufficiently flexible to pass from a reduced-volume configuration to an expanded configuration, and provided with at least one port,
a second pouch arranged so as to contain the first pouch, and provided with at least one hole, and
a sealing member tightly fastened onto the second pouch and designed to seal said port and said hole.

The invention also relates to a method of manufacturing an expandable intragastric balloon designed to be implanted inside the stomach of a patient for the treatment of obesity, wherein provisions are made for:
a step for manufacturing at least one first pouch that is sufficiently flexible to pass from a reduced-volume configuration to an expanded configuration, and that is provided with at least one port,
a step for assembling the first pouch together with a second pouch, provided with at least one hole, with the result being that the first pouch is contained inside the second pouch,
a step for manufacturing a sealing member designed to seal said port and said hole, and
a step for fastening the sealing member onto the second pouch in a substantially leak-proof manner.

### BACKGROUND OF THE INVENTION

Intragastric balloons used for the treatment of obesity generally comprise one or more concentric, flexible pouches that are made of the same material, such as silicone, and that are capable of passing from a reduced-volume or folded-over configuration to an expanded configuration, thereby imparting the balloon with its functional shape.

The pouches are thus often filled with an inflating fluid and, for this purpose, comprise a valve-type sealing member designed to ensure their leak tightness. For this purpose, the pouches are equipped with a port designed to be sealed by the sealing member, the latter generally being manufactured from the same material as the pouches and welded or glued together with the periphery of the hole.

Although it has certain advantages, particularly in terms of simplicity and economy of manufacture, the design of these intragastric balloons suffers nevertheless from several disadvantages, associated, in particular, with the technique of assembling the components (pouches and sealing member) by welding or gluing.

Thus, although it is relatively easy to weld or glue together materials of the same type, it may prove more tricky to perform this operation for different and incompatible materials for which it is difficult or even impossible to find a common welding or gluing material capable of ensuring that the balloon is perfectly leak-proof, while at the same time meeting other criteria (bio-compatibility, etc.).

Such being the case, it may turn out to be desirable to produce one of the components of the balloon, e.g., one of the pouches or the sealing member, with a new material, particularly if the latter has better properties (leak tightness, strength, etc.) for the application under consideration.

In a case such as this, it is critical to limit the impacts due to the use of this new material on the manufacture of the other balloon components and their assembly.

In the case of the intragastric balloons of the prior art, such impacts are difficult to prevent, in as much as the welding or gluing material must necessarily be compatible, on the one hand, with the material forming the sealing member and, on the other hand, with the material forming the pouches.

### SUMMARY OF THE INVENTION

Consequently, the features afforded by the invention aim to remedy the various disadvantages listed above and to propose a new, expandable intragastric balloon for the treatment of obesity, the components of which may be easily assembled while at the same time being manufactured from different materials.

Another feature of the invention aims to propose a new intragastric balloon whose design makes it possible to use a large range of materials for its manufacture.

Another feature of the invention aims to propose a new intragastric balloon whose manufacture is particularly simple and fast.

Another feature of the invention is to propose a new intragastric balloon provided with a sealing member whose leak tightness can be reliably reproduced.

Another feature of the invention aims to propose a new intragastric balloon whose overall leak tightness is improved.

Another feature of the invention aims to propose a new intragastric balloon having a generally good degree of mechanical strength.

Another feature of the invention aims to propose a new intragastric balloon whose structure makes it possible to improve the positioning of the balloon inside the stomach of the patient and to limit the undesirable effects of the implant on the functioning of the digestive system.

The features afforded by the invention also aim to propose a new method of manufacturing an expandable intragastric balloon that can be reproduced, and that is simple and quick to implement, while at the same time making it possible to obtain a balloon having an excellent degree of leak tightness.

Another feature of the invention aims to propose a new method of manufacturing an intragastric balloon that makes it possible to guarantee the leak tightness of the sealing member for the balloon, and to do so in a systematic and reproducible manner.

The features afforded by the invention are attained with the aid of an expandable intragastric balloon designed to be implanted inside the stomach of a patient for the treatment of obesity, and comprising:
a first pouch that is sufficiently flexible to pass from a reduced-volume configuration to an expanded configuration, and provided with at least one port,
a second pouch arranged so as to contain the first pouch, and provided with at least one hole, and
a sealing member fastened onto the second pouch in a leak-proof manner, and designed to seal said port and said hole,
   characterized in that the first and the second pouches are made of different, non-compatible materials and are assembled together with the aid of a fastening element designed to ensure the leak-proof fastening of the sealing member onto the first pouch, inside a passage defined by a neck extending from the port, by exerting a sufficient amount of pressure on said neck in order to pinch it between the sealing element and the fastening element.

The features afforded by the invention are also attained with the aid of a method for manufacturing an expandable intragastric balloon designed to be implanted inside the stomach of a patient for the treatment of obesity, wherein provisions are made for:
a step for manufacturing at least one first pouch that is sufficiently flexible to pass from a reduced-volume configuration to an expanded configuration, and that is provided with at least one port,
a step for assembling the first pouch together with a second pouch, provided with at least one hole, with the result being that the first pouch is contained inside the second pouch,
a step for manufacturing a sealing member designed to seal said port and said hole, and
a step for fastening the sealing member onto the second pouch in a substantially leak-proof manner,
   characterized in that, in order to assemble the first and the second pouch, the method comprises:
a step for mounting the sealing member inside a passage defined by a neck, extending from the port of the first pouch, and
a step for fastening the sealing member onto the first pouch with the aid of a suitable fastening element, by pinching said neck between the sealing member and the fastening element.

### BRIEF DESCRIPTION OF THE DRAWINGS

Other features and advantages of the invention will be more apparent after reading the following description and consulting the drawings provided in appendices, which are given purely for purpose of illustration and are in no way limiting, in which:

FIG. 1 is a cross-sectional view showing a first exemplary embodiment of single-pouch intragastric balloon in accordance with the invention, in its expanded configuration.

FIG. 2 is a cross-sectional view showing an exemplary embodiment of a double-pouch intragastric balloon in accordance with the invention, in its expanded configuration.

FIG. 3 is a cross-sectional view showing an exemplary embodiment of a double-pouch intragastric balloon in accordance with the invention, in its expanded configuration.

### DESCRIPTION OF THE INVENTION

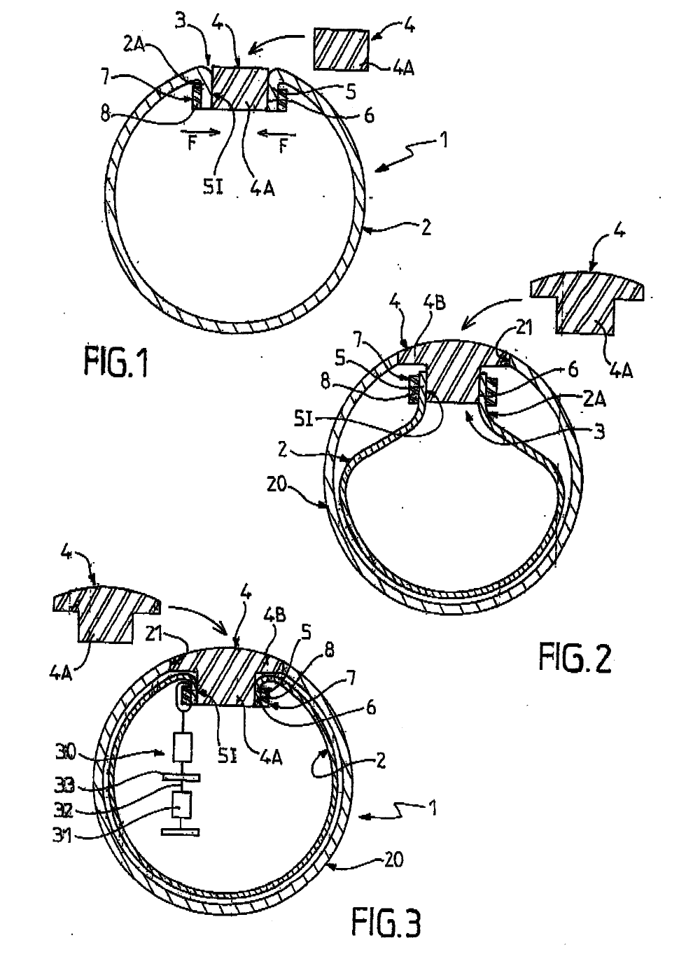

The intragastric balloon in accordance with the invention will now be described while referring to FIGS. 1-3.

The intragastric balloon 1 in accordance with the invention is designed to be implanted inside the stomach of a patient for the treatment of obesity.

The balloon 1 is expandable and, for this purpose, comprises at least one first pouch 2 that is sufficiently flexible to pass from a reduced-volume configuration to an expanded configuration. The reduced-volume configuration, for example, may correspond to a configuration in which the balloon 1 is in a folded-over position occupying a reduced volume, thereby facilitating the insertion of the balloon 1 into the esophagus.

As a matter of fact, implantation of the intragastric balloon 1 in accordance with the invention is generally performed, in a conventional manner well-known to those skilled in the art, by passing through the oral-esophageal route in its folded-over, compressed or loose form. The expansion, placement and release of the balloon occur at the end of the gastro-endoscopic operation, when the balloon 1 is correctly positioned inside the stomach of the patient.

As a matter of fact, it is in its expanded configuration that the balloon 1 will be able to occupy a considerable volume inside the gastric cavity (not shown), the so-called expanded configuration then imparting the balloon with its functional shape, i.e., the therapeutic operating shape of the balloon for the treatment of obesity. Thus, by occupying a portion of the internal volume of the stomach, the intragastric balloon according to the invention makes it possible to create a rapid sensation of fullness in the patient, which is accompanied by a reduction in the amount of food ingested.

According to the invention, the first pouch 2 comprises at least one port 3, which may result from the method of manufacturing the first pouch 2, or else be specifically made in said first pouch 2, for example, for the purpose of introducing an inflating fluid therein.

According to the invention, the balloon 1 also comprises a sealing member 4 for said first pouch 2, designed to seal the port 3. As a matter of fact, the first pouch 2 is preferentially designed to be filled with an inflating fluid, e.g., a gas or a liquid, and the sealing member 4 is designed to ensure the leak tightness of the first pouch 2, once the latter has inflated.

According to the invention, the first pouch 2 is provided with a neck 5, which extends from the port 3 in order to define a passage 6 between the interior and the exterior of the first pouch 2. The passage 6 is thus advantageously designed to receive the sealing member 4.

The neck 5 is advantageously formed by a folded-over section 2A of the wall of the first pouch 2, arranged so as to run substantially completely around the sealing member 4.

According to the invention, the balloon 1 comprises a fastening element 7 designed to ensure the fastening of the sealing member 4 onto the first pouch 2. For this purpose, the fastening element 7 is designed to exert pressure on the neck 5, which tends to close the latter onto the sealing member 4.

The fastening element 7 is designed, in particular, for exerting sufficient pressure on the neck 5 in order to pinch it between the sealing member 4 and the fastening element 7, thereby ensuring, on the one hand, the fastening of the sealing member 4 onto the first pouch 2 and, on the other hand, the leak tightness of the latter in the area of the port 3.

The intragastric balloon 1 thus designed therefore enables simple and reliable mounting of the sealing member 4 on the first pouch 2, even though these two elements are manufactured from different materials whose conventional assembly by gluing or by using weld lines would prove to be particularly difficult.

Mounting in this way thereby makes it possible to produce a strong assembly of the balloon components, and this is done without necessarily using multiple weld lines or gluing operations.

Advantageously, the neck 5 comprises an internal wall 5I, which defines the passage 6. The fastening element 7 is thus advantageously arranged so as to surround the neck 5 and exert a substantially even pressure over the circumference thereof, such that the internal wall 5I of the neck 5 conforms in shape substantially to the sealing member 4, in a leak-proof manner.

In a particularly advantageous way, the pressure exerted by the fastening element 7 is oriented in a centripetal direction (arrows F in FIG. 1).

Advantageously, the sealing member 4 includes a septum 4A having a self-sealing effect, which is arranged inside the passage 6 substantially opposite the fastening element 7. The neck 5 and the septum 4A are thus advantageously shaped so that the neck 5 substantially surrounds the septum 4A in its position of sealing the first pouch 2.

In a particularly advantageous way, the fastening element 7 is designed to sufficiently compress the septum 4A in order to ensure the leak tightness thereof, with respect to the fluids likely to be contained inside the first pouch 2. Thus, by exerting pressure on the neck 5, the fastening element 7 at the same time compresses the septum 4A in order to ensure its leak tightness.

Thus, the fastening element 7 advantageously and simultaneously provides a dual function:
on the one hand, it ensures the assembly of the sealing member 4 on the first pouch 2, and
on the other hand, it imparts the sealing member 4 with its own sealing function, by ensuring compression of the septum 4A.

For this reason, the pressure exerted by the fastening element 7 on the neck 5 will have to be sufficient to:
on the one hand, ensure the leak tightness between the neck 5 and the sealing member 4, and
on the other hand, ensure the leak tightness of the septum 4A.

The previously described assembly therefore makes it possible to limit the number of pieces required in order to provide the same functions.

In a particularly advantageous way, the fastening element 7 consists of a ring 8 preferably in the form of a cylindrical tube and arranged so as to surround the neck 5. The ring 8 is preferably substantially rigid and dimensioned to sufficiently compress the septum 4A in a centripetal direction so as to obtain a sufficient leak tightness thereof. The diameter of the ring 8, in particular, must be calculated with respect to the compression rate of the septum 4A that is required in order to obtain proper leak tightness.

In a particularly advantageous way, the first pouch 2 and the sealing member 4 are made from different materials, and preferentially from elastomers.

Several exemplary embodiments of the invention will now be described with reference to FIGS. 1 to 3.

According to a first exemplary embodiment of the invention shown in FIG. 1, the intragastric balloon 1 comprises only a single pouch 2 with a port 3 sealed by a sealing member 4. In this exemplary embodiment of the invention, the sealing member 4 is preferentially formed by the septum 4A, which becomes lodged inside the neck 5 which extends from the port 3 towards the interior of the pouch 2, thereby forming a re-entrant neck.

The pouch 2 is preferentially made of elastomer polyurethane, and therefore has good properties of flexibility and elasticity. As concerns the septum 4A, it is preferentially made of a silicone base. The fastening element 7, specifically the ring 8, thus surrounds the neck 5 and compresses the septum 4A, thereby ensuring, on the one hand, that the latter is leak-proof and, on the other hand, that it is fastened onto the pouch 2 in a leak-proof manner, by pinching the neck 5 between the septum 4A and the ring 8 (FIG. 1).

Advantageously, the septum 4A does not protrude outwardly from the pouch 2, thereby imparting the intragastric balloon 1 with an atraumatic property.

According to a second and third exemplary embodiment of the invention, shown in FIGS. 2 and 3, the intragastric balloon 1 advantageously comprises a second flexible pouch 20 arranged so as to contain the first pouch 2, thereby forming the outer casing of the balloon.

In a particularly advantageous way, and as shown in FIGS. 2 and 3, the fastening element 7 is designed to ensure the assembly of the first, inner pouch 2 with the second, outer pouch 20.

For this purpose, the second pouch 20 is advantageously provided with at least one hole 21, and the sealing member 4 is designed to seal said hole 21 in a substantially leak-proof manner. Thus, the sealing member preferably comprises a flange 4B designed to enable the leak-proof fastening of the sealing member 4 onto the second pouch 20, e.g., by gluing or welding the flange 4B onto the periphery of the hole 21.

Preferentially, the material forming the sealing member 4 is compatible with the material of which the second pouch 20 is made, at least in the area of the periphery of the hole 21, so as to enable the fastening of said sealing member 4 onto the second pouch 20, by welding or gluing.

The sealing member 4 is thus advantageously fastened directly onto the second pouch 20 by welding or gluing.

Preferentially, the sealing member 4 consists of a material having substantially the same chemical and physical properties as the material forming the second pouch 20.

Even more preferentially, the sealing member 4 and the second pouch 20 are made of the same material, such as silicone.

According to one possible exemplary embodiment of the invention, not shown in the figures, the sealing member 4 and the second pouch 20 are made integral with each other and form a one-piece unit, capable of being obtained by molding.

Advantageously, the flange 4B is arranged so as to cover the neck 5 of the first pouch 2 and the fastening element 7, in order to protect the assembly of the first pouch 2 with the sealing member 4 and prevent them from becoming disconnected, e.g., as a result of the movement of the intragastric balloon 1 inside the stomach. As a matter of fact, if the neck 5 and/or the fastening element 7 protrude outwardly from the balloon, the repeated movements of the balloon inside the stomach of the patient might cause the fastening element 7 to gradually loosen, which the flange 4B makes it possible to prevent.

The fastening element 7 thus provides a third function: to assemble the first and second pouches 2, 20, and to do so in a particularly reliable and reproducible manner, regardless of the type of materials forming said pouches 2, 20. With this assembly, it is thus possible to use two different and not necessarily compatible materials, in terms of welding or gluing, in order to produce the inner pouch and the outer casing, and to do so without any additional manufacturing constraint.

Preferentially, the first and second pouches 2, 20 are made of different, non-compatible materials, the first pouch 2 preferably being made of polyurethane, while the second pouch 20 is preferentially made of silicone.

Advantageously, the first, inner pouch 2 is made of a material having better leak-proof properties than the material forming the second, outer pouch 20.

Owing to the good leak-proof properties of the polyurethane, with respect to gases, it is possible to reduce the thickness of the first pouch 2, which has the direct effect of substantially reducing the overall dimensions of the balloon 1 in its reduced-volume configuration, thereby facilitating its implantation.

In the two exemplary embodiments shown in FIGS. 2 and 3, the sealing member 4 advantageously consists of a silicone valve, a portion of which consists of the silicone septum 4A, which is designed to become lodged inside the neck 5.

A sealing member 4 such as this thus allows an inflating needle to pass through the septum 4A, thereby enabling the filling of the first pouch 2, the self-sealing property of the sealing member 4, specifically the septum 4A, ensuring the leak tightness of the device when the inflating needle is withdrawn.

According to the third exemplary embodiment shown in FIG. 3, the neck 5 extends from the port 3 towards the interior of the first pouch 2, thereby forming a re-entrant neck. Owing to this configuration, the second pouch 20 advantageously conforms to the shape of the first pouch 2 when the latter is filled with the inflating fluid. The balloon then has a substantially compact and therefore mechanically stronger structure.

Quite obviously, it is also possible to produce an intragastric balloon in which the neck 5 of the first pouch extends from the port 3 towards the exterior of said first pouch so as to form an outwardly protruding neck (second exemplary embodiment shown in FIG. 2), and to do so without exceeding the scope of the invention.

In a particularly advantageous way, the intragastric balloon 1 comprises a ballasting means 30 designed to substantially weigh down the balloon 1 (FIG. 3).

Thus, when the first pouch 2 is filled only with gas, the balloon 1, due to its low weight, may have a tendency to rise up into the upper portion of the stomach, thereby impeding the penetration of food into the gastric cavity.

On the other hand, if the first pouch 2 is filled only with liquid, the intragastric balloon 1 risks being too heavy, and therefore poorly tolerated by the patient. The use of the ballasting means 30 thus constitutes a compromise making it possible to improve positioning of the balloon inside the stomach and at the same time eliminate a source of discomfort for the patient.

Advantageously, the ballasting means 30 consist of a plurality of solid and dense bodies 31 joined together by thread portions 32.

Preferentially, the ballasting means 30 also comprise spacers 30 arranged between two consecutive solid and dense bodies 31 so as to prevent shocks, and therefore undesirable noise. The spacers 33 are preferably made of elastomer, e.g., silicone.

The solid and dense bodies 31 are preferentially manufactured from a tungsten base, preferred, in particular, because of its bio-compatible property.

In a particularly advantageous way, and as shown in FIG. 3, the ballasting means 30 is preferably arranged inside the first pouch 2, and one of the ends of the thread joining the solid and dense bodies 31 together is firmly attached to the fastening element 7, which makes it possible to restrict the mobility of the ballasting means 30 inside the balloon 1.

The fastening element 7 may thereby advantageously provide a fourth function, namely that of a support and attachment means for the ballasting means 30.

The invention also relates to a method of manufacturing an expandable intragastric balloon 1 designed to be implanted inside the stomach of a patient for the treatment of obesity, wherein the steps are provided:
for manufacturing at least one first pouch 2, that is sufficiently flexible to pass from a reduced-volume configuration to an expanded configuration, and that is provided with at least one port 3, and
for manufacturing a sealing member 4 for said first pouch 2, designed to seal said port 3.

Advantageously, the step for manufacturing the first pouch 2 includes a sub-step wherein two hemispheres are manufactured, e.g., by thermoforming, the edges of which are then welded (or glued) so as to form a substantially spherical first pouch 2 equipped with a neck 5.

More precisely, the method for manufacturing the first pouch 2 comprises a step during which one or more sheets (e.g., two sheets) of a predetermined shape, made of a material substantially impermeable to gases, such as thermoplastic elastomer polyurethane, that have been pre-shaped, e.g., by thermoforming, so as to impart them with a hemispherical shape, are assembled together by welding or gluing along a peripheral weld line.

Each sheet may consist of a single polyurethane film, but preferably consists of several and, for example, two superimposed polyurethane films, said films being capable of being made integral with each other, or movable in relation to each other.

Preferentially, each sheet comprises an extension, of rectangular shape, for example. During assembly of the sheets, the extensions are superimposed over one another and welded or glued together along a peripheral weld (or glue) line, so as to form the neck 5 of the first pouch 2.

Quite obviously, the step for manufacturing the first pouch 2 may likewise result from another process, such as a heat-sealing process, and without thereby exceeding the scope of the invention.

According to the invention, the method next comprises a step (a) for pinching the neck 5 between the sealing member 4 and a suitable fastening element 7, on the one hand, so as to ensure the fastening of the sealing member 4 onto the first pouch 2 and, on the other hand, the leak tightness of the latter.

This method thus makes it possible to advantageously eliminate the conventional step of welding or gluing the sealing member 4 onto the first pouch 2, thereby accelerating the manufacturing operation for the balloon 1. Furthermore, this method is particularly advantageous in cases where the first pouch 2 and the sealing member 4 are made of non-compatible materials, i.e., materials that are difficult or even impossible to assemble by welding or gluing with any guarantee of quality.

In a particularly advantageous way, the method comprises a step (b) for turning over the neck 5 such that it is situated inside the first pouch 2 and forms a re-entrant neck. The turnover step (b) will be preferentially carried out before the above-mentioned two hemispheres are completely welded together, so as to retain access to the inside of the first pouch 2.

Advantageously, the method next includes a step (c) for mounting the sealing member 4 inside a passage 6 formed by the neck 5, followed by a step (d) for mounting a ring 8 around the neck 5, said ring 5 forming the fastening element 7, so as to surround the neck 5 and exert pressure on the circumference thereof, such that the internal wall 5I of the neck 5 conforms in shape substantially to the sealing member 4, in a leak-proof manner.

Advantageously, the method also comprises a step (e) for assembling the first pouch 2 with a second pouch 20, wherein the sealing member 4 is fastened onto the second pouch 20 in a substantially leak-proof manner. For this purpose, the assembling step (e) comprises a sub-step wherein the flange 4B of the sealing member 4 is welded or glued together with the periphery of the hole 21 provided in the second pouch 20.

A double-pouch design 2, 20 such as this thus makes it possible to make an intragastric balloon 1 that has improved mechanical strength, while at the same time retaining a flexible and elastic structure. In this way then, the first pouch preferably constitutes an inflation chamber and, in this regard, is designed to be filled with an inflating fluid, e.g., air, the second pouch 20 then constituting the protective outer casing of the balloon 1, the formation and, in particular, the deployment of which is controlled by the first pouch 2. Thus, as the first pouch 2 inflates, it will push out the wall of the second pouch 20, and will do so until said second pouch 20 attains its functional shape inside the stomach.

Owing to its particular design, the intragastric balloon 1 in accordance with the invention is capable of easily keeping up with the technical developments in the field, and particularly the developments relating to the materials used in the manufacture of the balloons.

Therefore, the invention makes it possible to produce a balloon whose components, particularly the pouches and sealing member, may be easily assembled while at the same time being structurally independent of one another and manufactured from separate, not necessarily compatible materials.

Another advantage of the intragastric balloon in accordance with the invention is that it benefits from good reproducibility, on the one hand, as concerns the assembly of its various components and, on the other hand, as concerns its functional capabilities. In particular, the fastening element 7 is dimensioned in order to reproducibly guarantee the sealing property of the sealing member 4, and this is accomplished by pre-calculating the rate of compression that the fastening element 7 must exert on the septum 4A in order to ensure the leak tightness thereof.

Another advantage of the intragastric balloon 1 in accordance with the invention comes from the fact that a single part, namely the fastening element 7, may be capable of providing several distinct functions, and in particular:
1) the assembly of the sealing member 4 on the first pouch 2,
2) the leak tightness of the sealing member 4, specifically the septum 4A,
3) the assembly of the first pouch 2 with the second pouch 20, and
4) the fastening of the ballasting means 30.

The invention finds one aspect of its industrial application in the design and manufacture of implantable devices for controlling obesity.

1. An expandable intragastric balloon designed to be implanted inside the stomach of a patient for the treatment of obesity, comprising:
(a) a first pouch that is sufficiently flexible to pass from a reduced-volume configuration to an expanded configuration, and provided with at least one port,
(b) a second pouch arranged so as to contain the first pouch, and provided with at least one hole, and
(c) a sealing member fastened onto the second pouch in a leak-proof manner, and designed to seal said port and said hole,
wherein the first and the second pouches are made of different, non-compatible materials and are assembled together with the aid of a fastening element designed to ensure the leak-proof fastening of the sealing member onto the first pouch, inside a passage defined by a neck extending from the port, by exerting a sufficient amount of pressure on said neck in order to pinch it between the sealing member and the fastening element.

2. The intragastric balloon of claim 1, wherein the sealing member and the second pouch are made of compatible materials, and wherein the sealing member is fastened onto the second pouch by welding or gluing.

3. The intragastric balloon of claim 1, wherein the sealing member comprises a flange enabling it to be fastened onto the periphery of the hole in a leak-proof manner, by gluing or welding the flange together with the periphery of the hole.

4. The intragastric balloon of claim 1, wherein the sealing member comprises a septum arranged inside the passage substantially opposite the fastening element.

5. The intragastric balloon of claim 1, wherein the fastening element is designed to sufficiently compress the sealing member, particularly the septum, in order to ensure the leak tightness thereof, with respect to the fluids likely to be contained inside the first pouch.

6. The intragastric balloon of claim 1, wherein the neck extends from the port towards the interior of the first pouch.

7. The intragastric balloon of claim 1, wherein the neck comprises an internal wall defining said passage and the fastening element is arranged so as to surround the neck and to exert pressure on the circumference thereof, such that the internal wall of the neck conforms in shape substantially to the sealing member in a leak-proof manner.

8. The intragastric balloon of claim 1, wherein the fastening element comprises a ring.

9. The intragastric balloon of claim 1, wherein the first pouch is made of polyurethane, and the second pouch is made of silicone.

10. The intragastric balloon of claim 1, wherein the sealing member comprises a silicone valve.

11. The intragastric balloon of claim 1, further comprising a ballasting means designed to substantially weigh down the balloon.

12. The intragastric balloon of claim 11, wherein the ballasting means comprises a plurality of solid and dense bodies joined together by thread portions.

13. The intragastric balloon of claim 12, wherein the ballasting means comprise spacers arranged between two consecutive bodies so as to prevent shocks.

14. The intragastric balloon of claim 10, wherein the ballasting means is supported by the fastening element.

15. The intragastric balloon of claim 12, wherein one of the ends of the thread joining together the bodies is attached to the fastening element.

16. The intragastric balloon of claim 11, wherein the ballasting means is arranged inside the first pouch.

17. A method of manufacturing an expandable intragastric balloon designed to be implanted inside the stomach of a patient for the treatment of obesity, comprising:
(a) manufacturing at least one first pouch that is sufficiently flexible to pass from a reduced-volume configuration to an expanded configuration, and that is provided with at least one port;
(b) assembling the first pouch together with a second pouch, provided with at least one hole, with the result being that the first pouch is contained inside the second pouch;
(c) manufacturing a sealing member designed to seal said port and said hole; and
(d) fastening the sealing member onto the second pouch in a substantially leak-proof manner,
wherein, in order to assemble the first and the second pouch, the method comprises:
(e) mounting the sealing member inside a passage defined by a neck extending from the port of the first pouch,
(f) fastening the sealing member onto the first pouch with the aid of a suitable fastening element, by pinching said neck between the sealing member and the fastening element.

18. The method of claim 17, wherein the step for fastening the sealing member onto the second pouch is carried out by welding or gluing.

19. The method of claim 17, wherein the sealing member comprises a flange and the method comprises a sub-step wherein said flange is welded or glued about the periphery of the hole.

20. The method of claim 17, further comprising the step of turning over the neck such that it is situated inside the first pouch.

21. The intragastric balloon of claim 2, wherein the sealing member comprises a flange enabling it to be fastened onto the periphery of the hole in a leak-proof manner by gluing or welding the flange about the periphery of the hole.

22. The intragastric balloon of claim 2, wherein the sealing member comprises a septum arranged inside the passage substantially opposite the fastening element.

23. The intragastric balloon of claim 3, wherein the sealing member comprises a septum arranged inside the passage substantially opposite the fastening element.

24. The intragastric balloon of claim 14, wherein one of the ends of the thread joining together the bodies is attached to the fastening element.

25. The method of claim 18, wherein the sealing member comprises a flange and the method comprises a sub-step wherein said flange is welded or glued about the periphery of the hole.
